(19) United States
(12) Patent Application Publication
(10) Pub. No.: US 2022/0319678 A1
(43) Pub. Date: Oct. 6, 2022

(54) METHODS, SYSTEMS, AND COMPUTER PROGRAM PRODUCTS FOR PROCESSING MEDICAL CLAIM DENIALS USING AN ARTIFICIAL INTELLIGENCE ENGINE

(71) Applicant: Change Healthcare Holdings LLC, Nashville, TN (US)

(72) Inventors: Jaison Harada, Glendora, NJ (US); Nanette Mapes, Macomb, MI (US); Tina M. Eller, Wilmington, DE (US)

(21) Appl. No.: 17/218,868

(22) Filed: Mar. 31, 2021

(51) Int. Cl. G16H 40/20; G06Q 40/08; G06Q 20/42; H04M 3/523

(52) U.S. Cl. CPC G16H 40/20; G06Q 40/08; G06Q 20/42; H04M 3/5233

(57) ABSTRACT

A method includes receiving a plurality of payment denials for medical claims from a plurality of payors; assigning, using an artificial intelligence engine, priorities to the plurality of payment denials, respectively; assigning, using the artificial intelligence engine, the plurality of payment denials to a plurality of agents responsible for obtaining payment approvals for the medical claims for which the payment denials have been received, respectively, based on the priorities.

### FIELD

The present inventive concepts relate generally to health care systems and services and, more particularly, to the use of artificial intelligence systems that can be used by health care providers for processing of medical claim denials.

### BACKGROUND

Health care service providers have patients that pay for their care using a variety of different payors. For example, a medical facility or practice may serve patients that pay by way of different insurance companies including, but not limited to, private insurance plans, government insurance plans, such as Medicare, Medicaid, and state or federal public employee insurance plans, and/or hybrid insurance plans, such as those that are sold through the Affordable Care Act. When providers submit claims to the payors for payment, however, the claims can be denied in whole or in part for a variety of different reasons. Some of these denials may be overcome if a provider can understand the reason for the denial and can remedy any deficiency in the originally submitted claim. Unfortunately, many denied claims are never overcome resulting in lost revenue for providers and/or more out of pocket expense for patients.

### SUMMARY

According to some embodiments of the inventive concept, a method comprises receiving a plurality of payment denials for medical claims from a plurality of payors; assigning, using an artificial intelligence engine, priorities to the plurality of payment denials, respectively; assigning, using the artificial intelligence engine, the plurality of payment denials to a plurality of agents responsible for obtaining payment approvals for the medical claims for which the payment denials have been received, respectively, based on the priorities.

In other embodiments, assigning, using the artificial intelligence engine, the priorities comprises: assigning, using the artificial intelligence engine, the priorities to the plurality of payment denials, respectively, based on projected values associated with the plurality of payment denials, respectively.

In still other embodiments, the method further comprises determining, using the artificial intelligence engine, probabilities of obtaining payment approvals for the medical claims for which the payment denials have been received, respectively; and estimating, using the artificial intelligence engine, payment amounts for the medical claims for which the payment denials have been received, respectively.

In still other embodiments, the method further comprises determining the projected values associated with the plurality of payment denials based on the probabilities of obtaining payment approvals that have been determined and the payment amounts that have been estimated, respectively.

In still other embodiments, assigning, using the artificial intelligence engine, the plurality of payment denials to the plurality of agents comprises: assigning, using the artificial intelligence engine, the plurality of payment denials to the plurality of agents based on the priorities and characteristics associated with each of the plurality of agents.

In still other embodiments, the characteristics associated with each of the plurality of agents comprises one or more of a plurality of skillsets, an availability, and a location.

In still other embodiments, each of the plurality of skillsets is associated with at least one of a plurality of claim adjustment reason codes used by the plurality of payors.

In still other embodiments, the method further comprises determining for one of the plurality of agents times taken for obtaining payment approvals for the medical claims that have been assigned to the one of the plurality of agents, respectively for different ones of the plurality of skillsets.

In still other embodiments, the plurality of skillsets comprises a first skillset associated with provider credentialing, a second skillset associated with treatment pre-authorization, a third skillset associated with medical coding, a fourth skillset associated with payor plan eligibility, a fifth skillset associated with payor underpayment, a sixth skillset associated with documentation requests, a seventh high level general skillset, an eight medium level general skillset, and/or a ninth low level general skillset.

In still other embodiments, the availability is based on a number of the plurality of payment denials assigned to the respective one of the plurality of agents.

In still other embodiments, the location identifies a time zone for the respective one of the plurality of agents.

In some embodiments of the inventive concept, a system comprises a processor and a memory coupled to the processor and comprising computer readable program code embodied in the memory that is executable by the processor to perform operations comprising: receiving a plurality of payment denials for medical claims from a plurality of payors; assigning, using an artificial intelligence engine, priorities to the plurality of payment denials, respectively; and assigning, using the artificial intelligence engine, the plurality of payment denials to a plurality of agents responsible for obtaining payment approvals for the medical claims for which the payment denials have been received, respectively, based on the priorities.

In further embodiments, assigning, using the artificial intelligence engine, the priorities comprises: assigning, using the artificial intelligence engine, the priorities to the plurality of payment denials, respectively, based on projected values associated with the plurality of payment denials, respectively.

In still further embodiments, the operations further comprise: determining, using the artificial intelligence engine, probabilities of obtaining payment approvals for the medical claims for which the payment denials have been received, respectively; and estimating, using the artificial intelligence engine, payment amounts for the medical claims for which the payment denials have been received, respectively.

In still further embodiments, the operations further comprise: determining the projected values associated with the plurality of payment denials based on the probabilities of obtaining payment approvals that have been determined and the payment amounts that have been estimated, respectively.

In still further embodiments, assigning, using the artificial intelligence engine, the plurality of payment denials to the plurality of agents comprises: assigning, using the artificial intelligence engine, the plurality of payment denials to the plurality of agents based on the priorities and characteristics associated with each of the plurality of agents.

In some embodiments of the inventive concept, a computer program product comprises a non-transitory computer readable storage medium comprising computer readable program code embodied in the medium that is executable by a processor to perform operations comprising: receiving a plurality of payment denials for medical claims from a plurality of payors; assigning, using an artificial intelligence engine, priorities to the plurality of payment denials, respectively; and assigning, using the artificial intelligence engine, the plurality of payment denials to a plurality of agents responsible for obtaining payment approvals for the medical claims for which the payment denials have been received, respectively, based on the priorities.

In other embodiments, assigning, using the artificial intelligence engine, the priorities comprises: assigning, using the artificial intelligence engine, the priorities to the plurality of payment denials, respectively, based on projected values associated with the plurality of payment denials, respectively. The operations further comprise determining, using the artificial intelligence engine, probabilities of obtaining payment approvals for the medical claims for which the payment denials have been received, respectively; and estimating, using the artificial intelligence engine, payment amounts for the medical claims for which the payment denials have been received, respectively.

In still other embodiments, the operations further comprise: determining the projected values associated with the plurality of payment denials based on the probabilities of obtaining payment approvals that have been determined and the payment amounts that have been estimated, respectively.

In still other embodiments, assigning, using the artificial intelligence engine, the plurality of payment denials to the plurality of agents comprises: assigning, using the artificial intelligence engine, the plurality of payment denials to the plurality of agents based on the priorities and characteristics associated with each of the plurality of agents.

It is noted that aspects described with respect to one embodiment may be incorporated in different embodiments although not specifically described relative thereto. That is, all embodiments and/or features of any embodiments can be combined in any way and/or combination. Moreover, other methods, systems, articles of manufacture, and/or computer program products according to embodiments of the inventive concept will be or become apparent to one with skill in the art upon review of the following drawings and detailed description. It is intended that all such additional systems, methods, articles of manufacture, and/or computer program products be included within this description, be within the scope of the present inventive subject matter, and be protected by the accompanying claims.

### BRIEF DESCRIPTION OF THE DRAWINGS

Other features of embodiments will be more readily understood from the following detailed description of specific embodiments thereof when read in conjunction with the accompanying drawings, in which:

FIG. 1 is a block diagram that illustrates a communication network including an Artificial Intelligence (AI) assisted medical claim denial processing system in accordance with some embodiments of the inventive concept;

FIG. 2 is a block diagram of the AI assisted medical claim denial processing system of FIG. 1 in accordance with some embodiments of the inventive concept;

FIGS. 3-5 are flowcharts that illustrate operations for processing medical claim denials using the AI assisted medical claim denial processing system of FIG. 1 in accordance with some embodiments of the inventive concept;

FIGS. 6A and 6B are charts that illustrate prioritization methodologies for processing medical claim denials using the AI assisted medical claim denial processing system of FIG. 1 in accordance with some embodiments of the inventive concept;

FIG. 7 is a flowchart that illustrates further operations for processing medical claim denials using the AI assisted medical claim denial processing system of FIG. 1 in accordance with some embodiments of the inventive concept;

FIG. 8 is a data processing system that may be used to implement one or more servers in the AI assisted medical claim denial processing system of FIG. 1 in accordance with some embodiments of the inventive concept; and

FIG. 9 is a block diagram that illustrates a software/hardware architecture for use in the AI assisted medical claim denial processing system of FIG. 1 in accordance with some embodiments of the inventive concept.

### DETAILED DESCRIPTION

In the following detailed description, numerous specific details are set forth to provide a thorough understanding of embodiments of the present inventive concept. However, it will be understood by those skilled in the art that the present invention may be practiced without these specific details. In some instances, well-known methods, procedures, components and circuits have not been described in detail so as not to obscure the present inventive concept. It is intended that all embodiments disclosed herein can be implemented separately or combined in any way and/or combination. Aspects described with respect to one embodiment may be incorporated in different embodiments although not specifically described relative thereto. That is, all embodiments and/or features of any embodiments can be combined in any way and/or combination.

Embodiments of the inventive concept are described herein in the context of a medical claim denial processing system that includes a machine learning engine and an artificial intelligence (AI) engine. It will be understood that embodiments of the inventive concept are not limited to a machine learning implementation of the prediction engine and other types of AI systems may be used including, but not limited to, a multi-layer neural network, a deep learning system, a natural language processing system, and/or computer vision system Moreover, it will be understood that the multi-layer neural network is a multi-layer artificial neural network comprising artificial neurons or nodes and does not include a biological neural network comprising real biological neurons.

Some embodiments of the inventive concept stem from a realization that medical claim payment denials by payors, such as insurance companies, may result in lost revenue for providers and/or increased costs for patients that could be avoided if the denials were overcome. Embodiments of the inventive concept may provide an Artificial Intelligence (AI) medical claim denial processing system that may receive payment denials for medical claims from a plurality of payors and may organize the payment denials by assigning priorities to them. The payment denials may be organized in a variety of ways. For example, the payment denials may be organized in order of the probability of overturning the denial, in order of the expected payment if the denial is overturned, in order of age or days in which to file a request for reconsideration of the denial, or in order of projected value. According to some embodiments of the inventive concept, projected value may be based on a combination of the probability of overturning the denial and the expected payment if the denial is overturned. For example, projected value of a medical claim payment denial may be the product of the probability of overturning the denial and the expected payment if the denial is overturned. In some embodiments, an AI engine may be used in determining the probabilities of overturning the medical claim payment denials along with the expected payments if the denials are overturned. A medical claim payment denial may be assigned to an agent who is responsible for obtaining a payment approval (i.e., getting the payment denial overturned). These agents may develop skills that allow them to be more successful in obtaining payment approvals for medical claims related to certain types of subject matter or non-payment reasons relative to medical claims related to other types of subject matter or non-payment reasons. The AI engine may, in some embodiments, be used to assign the prioritized medical claim payment denials to the agents based on the agents' characteristics. For example, each agent may have characteristics associated therewith comprising one or more skillsets, an availability (e.g., how many claim denials does the agent have currently pending in a work queue), and a location of the agent. The skillsets may include, but are not limited to, a first skillset associated with provider credentialing, a second skillset associated with treatment pre-authorization, a third skillset associated with medical coding, a fourth skillset associated with payor plan eligibility, a fifth skillset associated with payor underpayment, a sixth skillset associated with documentation requests, a seventh high level general skillset, an eight medium level general skillset, and/or a ninth low level general skillset.

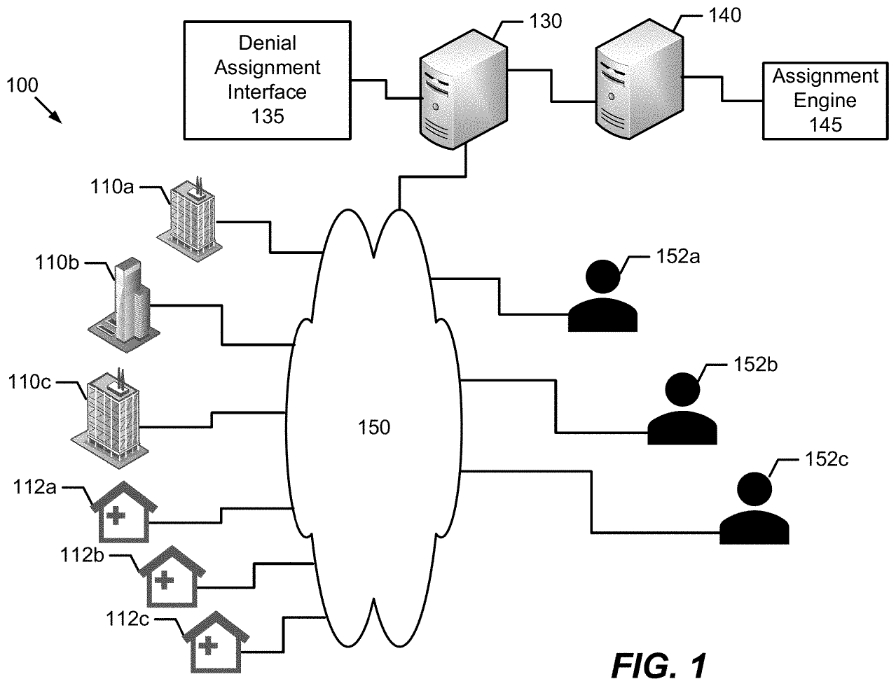

Referring to FIG. 1, a communication network 100 including an AI assisted medical claim denial processing system, in accordance with some embodiments of the inventive concept, comprises a plurality of medical claim payor sites 110a, 110b, and 110c, which may process medical claims for payment submitted by, for example, health care service providers. The health care provider facilities or practices 112a, 112b, and 112c may represent various types of organizations that are used to deliver health care services to patients, which are referred to generally herein as “providers.” The providers may include, but are not limited to, hospitals, medical practices, mobile patient care facilities, diagnostic centers, lab centers, and the like. The providers 112a, 112b, and 112c may operate by providing health care services for patients and then invoicing one or more payors 110a, 110b, and 110c for the services rendered. The payors may include, but are not limited to, private insurance plans, government insurance plans (e.g., Medicare, Medicaid, state or federal public employee insurance plans), hybrid insurance plans (e.g., Affordable Care Act plans), private medical cost sharing plans, and the patients themselves.

According to some embodiments of the inventive concept, providers 112a, 112b, and 112c may access the AI assisted medical claim denial processing system to allow them to evaluate and process denied medical claims and to resubmit them to the payor 110a, 110b, and 110c with a response that is designed to persuade the payor 110a, 110b, and 110c to withdraw the denial and pay the claim in full or in part. The AI assisted medical claim denial processing system may include an assignment engine interface server 130, which includes a denial assignment interface module 135 to facilitate the transfer of medical claim information between the respective providers 112a, 112b, and 112c, and an assignment engine server 140, which includes an assignment engine module 145. The assignment engine server 140 and assignment engine module 145 may be configured to receive medical claim denials from the payors 110a, 110b, and 110c by way of the assignment engine interface server 130 and denial assignment interface module 135. The denial assignment interface module 135 in conjunction with the assignment engine module 145 may be further configured to prioritize the claim denials received from the various payors 110a, 110b, and 110c for a particular provider 112a, 112b, and 112c and to intelligently assign these denied claims to a plurality of agents 152a, 152b, and 152c for evaluating the denied claims and submitting a response to the appropriate payor in an attempt to get the denial overturned or withdrawn. The denied claims may be assigned to the plurality of agents 152a, 152b, and 152c based on the agents' skillsets, availability, and/or location.

It will be understood that the division of functionality described herein between the assignment engine server 140/assignment engine module 145 and the assignment engine interface server 130/denial assignment interface module 135 is an example. Various functionality and capabilities can be moved between the assignment engine server 140/assignment engine module 145 and the assignment engine interface server 130/denial assignment interface module 135 in accordance with different embodiments of the inventive concept. Moreover, in some embodiments, the assignment engine server 140/assignment engine module 145 and the assignment engine interface server 130/denial assignment interface module 135 may be merged as a single logical and/or physical entity.

A network 150 couples the payors 110a, 110b, and 110c and the providers 112a, 112b, and 112c to the assignment engine interface server 130/denial assignment interface module 135. The network 150 may be a global network, such as the Internet or other publicly accessible network. Various elements of the network 150 may be interconnected by a wide area network, a local area network, an Intranet, and/or other private network, which may not be accessible by the general public. Thus, the communication network 150 may represent a combination of public and private networks or a virtual private network (VPN). The network 150 may be a wireless network, a wireline network, or may be a combination of both wireless and wireline networks.

The AI assisted medical claim denial processing service provided through the assignment engine interface server 130, denial assignment interface system module 135, assignment engine server 140, and assignment engine module 145, in some embodiments, may be embodied as a cloud service. For example, providers 112a, 112b, and 112c may integrate their claims submission systems with the AI assisted medical claim denial processing service and access the service as a Web service. In some embodiments, the AI assisted medical claim denial processing service may be implemented as a Representational State Transfer Web Service (RESTful Web service). The denial assignment interface system module 135 may further provide an interface for communicating the prioritized denied claims along with the assignment of the denied claims to the agents generated by the assignment engine server 140/assignment engine module 145 to, for example, a health care practice or facility manager. The interface may be embodied in a variety of ways including, but not limited to, an Application Programming Interface (API), one or more tables, one or more graphs/charts, a screen with one or more panes of text and/or graphic information, or the like. The results with respect to characteristics of the claims denials that are overturned and payment received may allow providers to make improvement to their claims generation process to reduce the likelihood of future claims being denied.

Although FIG. 1 illustrates an example communication network including an AI assisted medical claim denial processing system, it will be understood that embodiments of the inventive subject matter are not limited to such configurations, but are intended to encompass any configuration capable of carrying out the operations described herein.

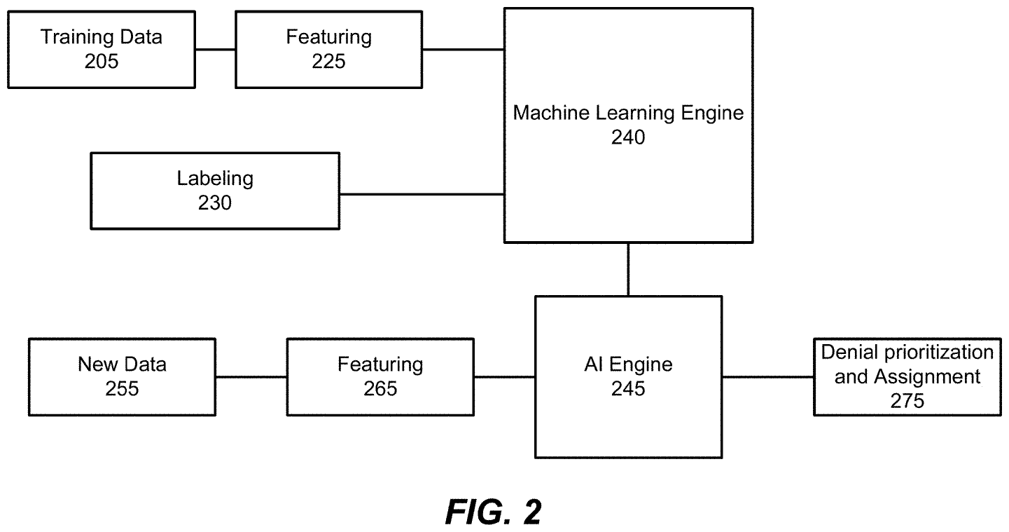

FIG. 2 is a block diagram of the assignment engine module 145 used in the AI assisted medical claim denial processing system in accordance with some embodiments of the inventive concept. As shown in FIG. 2, the assignment engine module 145 may include both training modules and modules used for processing new data on which to prioritize claim denials and to assign these denied claims to agents. The modules used in the training portion of the assignment engine module 145 include the training data 205, the featuring module 225, the labeling module 230, and the machine learning engine 240. The training data 205 may comprise information associated with prioritizing denied claims and assigning these claims to agents who re-submit the claims with additional information, as appropriate, in an attempt to get the denials overturned and withdrawn and the claims paid in full or in part. In some embodiments of the inventive concept, the training data 205 may comprise historical information indicative of the likelihood of various payors to reconsider denied claims or claim lines, which may be based on claim subject matter and the typical amounts recovered when a payor does reconsider a claim denial. The training data 205 may also include characteristics of the agents including, but not limited to, skillsets, availability, and/or location. The featuring module 225 is configured to identify the individual independent variables that are used by the assignment engine module 145 to prioritize the denied claims and to make assignments to the agents, which may be considered a dependent variable. For example, the training data 205 may be generally unprocessed or formatted and include extra information in addition to medical claim information, payor information, and agent information. For example, the medical claim data may include account codes, business address information, and the like, which can be filtered out by the featuring module 225. The features extracted from the training data 205 may be called attributes and the number of features may be called the dimension. The labeling module 230 may be configured to assign defined labels to the training data and to the prioritized denied claims and agent assignments to ensure a consistent naming convention for both the input features and the generated outputs. The machine learning engine 240 may process both the featured training data 205, including the labels provided by the labeling module 230, and may be configured to test numerous functions to establish a quantitative relationship between the featured and labeled input data and the generated outputs. The machine learning engine 240 may use modeling techniques to evaluate the effects of various input data features on the generated outputs. These effects may then be used to tune and refine the quantitative relationship between the featured and labeled input data and the generated outputs. The tuned and refined quantitative relationship between the featured and labeled input data generated by the machine learning engine 240 is output for use in the AI engine 245. The machine learning engine 240 may be referred to as a machine learning algorithm.

The modules used for processing new data on which to prioritize denied claims and to make agent assignments include the new data 255, the featuring module 265, the AI engine module 245, and the denial prioritization and assignment module 275. The new data 255 may be the same data/information as the training data 205 in content and form except the data will be used for prioritizing presently denied claims and assigning these claims to agents. Likewise, the featuring module 265 performs the same functionality on the new data 255 as the featuring module 225 performs on the training data 205. The AI engine 245 may, in effect, be generated by the machine learning engine 240 in the form of the quantitative relationship determined between the featured and labeled input data and the generated outputs. The AI engine 245 may, in some embodiments, be referred to as an AI model. The AI engine 245 may be configured to output generated priorities and agent assignments via the denial prioritization and assignment module 275. The denial prioritization and assignment module 275 may be configured to communicate the prioritization of the denied claims and/or the assignment of denied claims to agents in a variety of ways including tables, spreadsheets, or the like. The generated output may further highlight various characteristics of the denied claims and/or the agents that may have been impactful in the prioritization and/or the agent assignment or may have had little impact in the prioritization and/or the agent assignment.

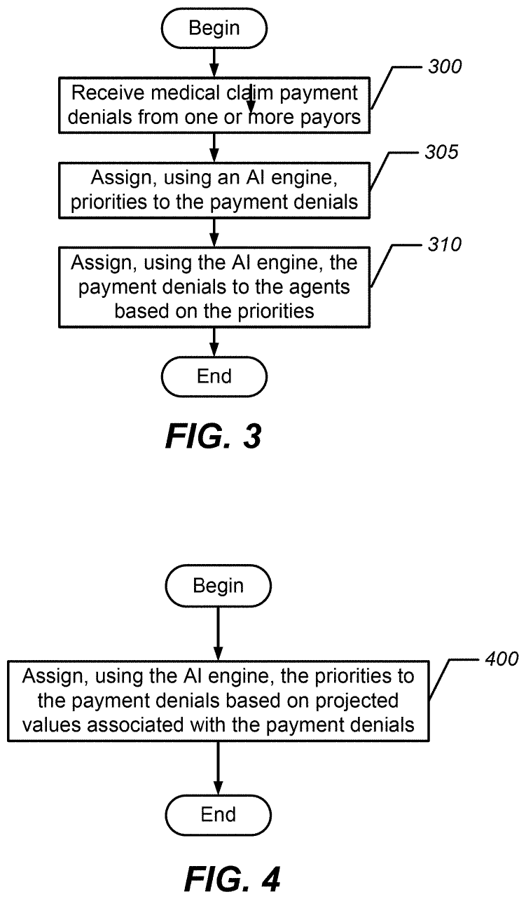

FIGS. 3-5 are flowcharts that illustrate operations for processing medical claim denials using the AI assisted medical claim denial processing system of FIG. 1 in accordance with some embodiments of the inventive concept. Referring now to FIG. 3, operations begin at block 300 where medical claim payment denials are received from one or more payors 110a, 110b, and 110c. A denied claim is a claim in which payment is denied by a payor. A denied claim may be a denial of an entire claim or a denial or one or more lines within a claim (payment is approved for some lines and denied for one or more other lines) in accordance with various embodiments of the inventive concept. The claims are then assigned priorities at block 305 using the AI engine 245. The AI engine is then used at block 310 to assign the denied claims or payment denials to one or more agents based on the priorities determined at block 305. Referring now to block 400, as described above, the denied claims or payment denials may be prioritized in a variety of ways including, but not limited to, in order of the probability of overturning the denial, in order of the expected payment if the denial is overturned, in order of age or days in which to file a request for reconsideration of the denial, or in order of projected value. Referring now to FIG. 4, according to some embodiments of the inventive concept, the AI engine 245 may use projected value as a basis for assigning the priorities to the denied claims or payment denials at block 400.

Referring now to FIG. 5, example embodiments for determining projected value begin at block 500 in which the AI engine 245 determines the probabilities of obtaining payment approvals for the various denied claims or payment denials. At block 505, the AI engine estimates the payment amounts for the medical claims for which the payment denials have been received. The projected values of each of the denied claims or payment denials may then be determined based on probability of obtaining payment approval (i.e., overturning the denial) and the estimated payment amount. In some embodiments, the projected value may be determined by computing the product of the probability of obtaining payment approval and the estimated payment amount.

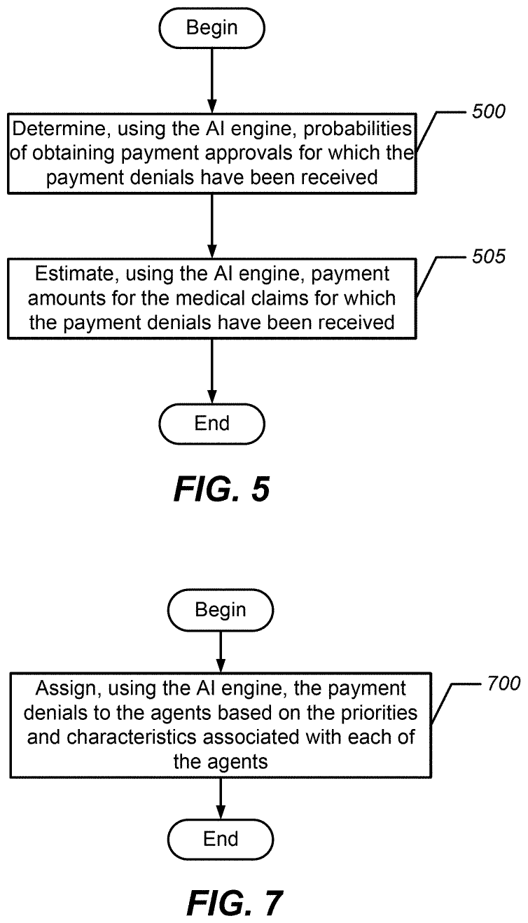

FIGS. 6A and 6B are charts that illustrate prioritization methodologies for processing medical claim denials using the AI assisted medical claim denial processing system of FIG. 1 in accordance with some embodiments of the inventive concept. In the example shown in FIG. 6A, claim denials are organized in order of the probability of overturning the denial and obtaining payment from the payor. For each denied claim, the probability of overturning the denial, the expected payment, the number of days left for which the denial may be appealed to the payor for reconsideration, and the projected value are shown. The projected value is computed as the product of the probability of overturning the claim denial decision and the expected payment if the denial is overturned. As shown in FIG. 6A, the highest priority denied claim has a projected value of only $1000 with several other lower priority denied claims having higher projected values. FIG. 6B illustrates the denied claims of FIG. 6A prioritized according to expected value. In this example, denied claim 9 has an expected value of $3000. Thus, even though the probability that the denial of claim 9 will be overturned is only 60%, due to a large expected payment of $5000, the expected value is the highest of all the denied claims at $3000. By prioritizing the denied claims according to expected value, the provider may increase the return on appealing denied claims to the payors.

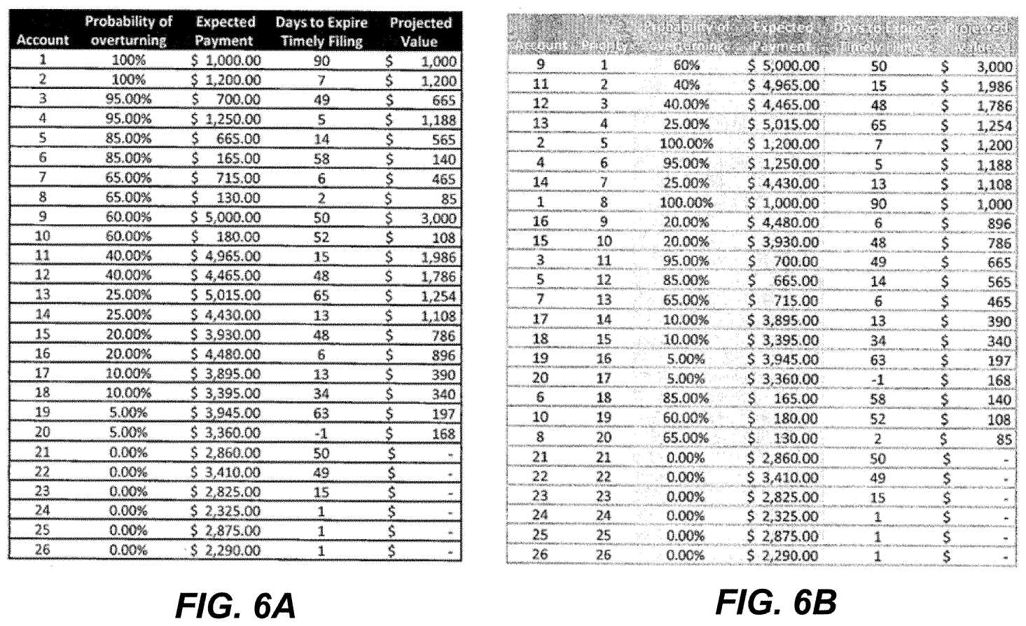

Referring now to FIG. 7, operations for assigning the denied claims to a plurality of agents begin at block 700 where the AI engine 245 assigns the denied claims or payment denials to the agents based on the priorities assigned to the claims and the characteristics associated with each of the agents. In some embodiments, the characteristics associated with each of the agents may comprise one or more skillsets, an availability, and/or a location. The skillsets may include, but are not limited to, a first skillset associated with provider credentialing (e.g., does the provider have the proper credentials to perform and bill for the medical service), a second skillset associated with treatment pre-authorization (e.g., certain medical procedures require pre-authorization of treatment before the treatment is performed), a third skillset associated with medical coding, a fourth skillset associated with payor plan eligibility (e.g., the patient may not be covered under the health care plan), a fifth skillset associated with payor underpayment, a sixth skillset associated with documentation requests, a seventh high level general skillset, an eight medium level general skillset, and/or a ninth low level general skillset. These skillsets may overlap in that an agent with a high-level general skillset may be qualified to work on a denied claim having subject matter associated with a medium level or low level skill set. In some embodiments, a skillset may be associated with one or more claim adjustment reason code used by one or more of the payors. The availability of an agent may refer to a current workload of the agent (i.e., the number of denied claims currently assigned to the agent or other assignments being handled by the agent). The location of the agent may refer to a particular geographic region or time zone in which the agent works. For example, it may be desirable to assign an agent to denied claims associated with a payor that is in a same or nearby time zone as the payor to facilitate communication between the payor and the agent. In some embodiments, the AI assisted medical claim denial processing system may track the times agents take in obtaining payment approvals for the medical claims that have been assigned to them for different ones of the plurality of skillsets to evaluate which skillsets an agent is able to use most effectively and/or determine which types of medical claim denials are more or less difficult to overturn. Such information may be used in determining probability of payment, expected value of that payment, and/or the effectiveness of various agents using different skillsets.

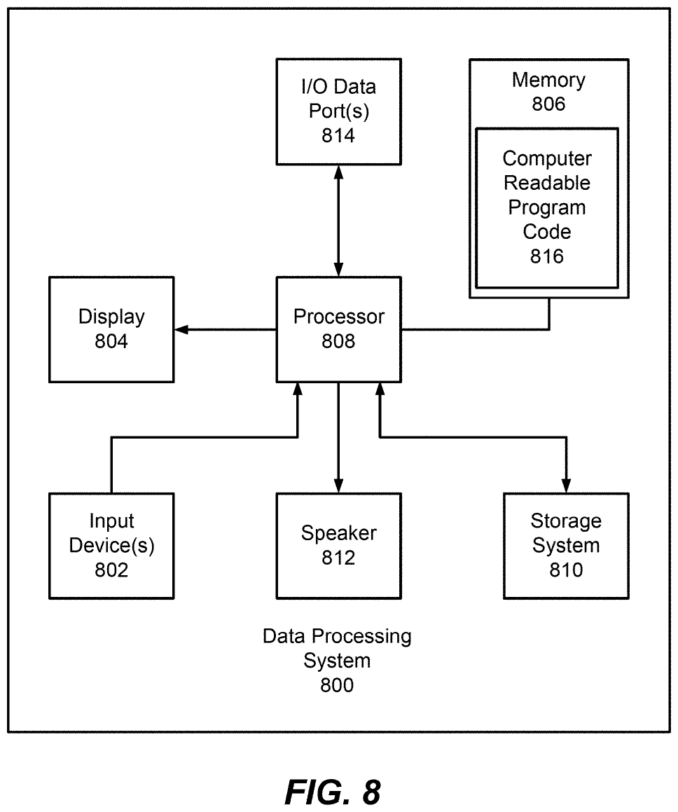

Referring now to FIG. 8, a data processing system 800 that may be used to implement the assignment engine server 140 of FIG. 1, in accordance with some embodiments of the inventive concept, comprises input device(s) 802, such as a keyboard or keypad, a display 804, and a memory 806 that communicate with a processor 808. The data processing system 800 may further include a storage system 810, a speaker 812, and an input/output (I/O) data port(s) 814 that also communicate with the processor 808. The processor 808 may be, for example, a commercially available or custom microprocessor. The storage system 810 may include removable and/or fixed media, such as floppy disks, ZIP drives, hard disks, or the like, as well as virtual storage, such as a RAMDISK. The I/O data port(s) 814 may be used to transfer information between the data processing system 800 and another computer system or a network (e.g., the Internet). These components may be conventional components, such as those used in many conventional computing devices, and their functionality, with respect to conventional operations, is generally known to those skilled in the art. The memory 806 may be configured with computer readable program code 816 to facilitate AI assisted medical claim denial processing according to some embodiments of the inventive concept.

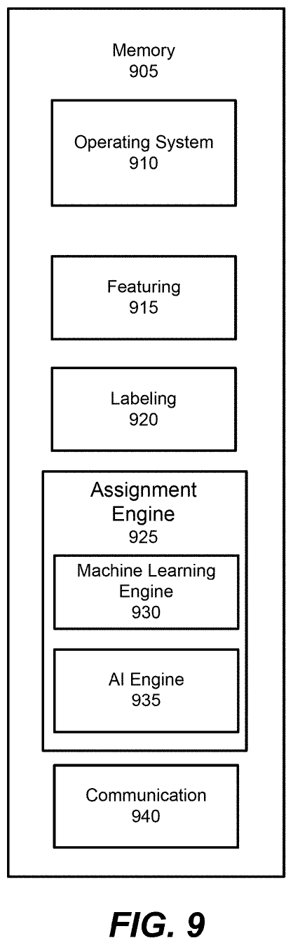

FIG. 9 illustrates a memory 905 that may be used in embodiments of data processing systems, such as the assignment engine server 140 of FIG. 1 and the data processing system 800 of FIG. 8, respectively, to facilitate AI assisted medical claim denial processing according to some embodiments of the inventive concept. The memory 905 is representative of the one or more memory devices containing the software and data used for facilitating operations of the assignment engine server 140 and assignment engine module 145 as described herein. The memory 905 may include, but is not limited to, the following types of devices: cache, ROM, PROM, EPROM, EEPROM, flash, SRAM, and DRAM. As shown in FIG. 9, the memory 905 may contain five or more categories of software and/or data: an operating system 910, a featuring module 915, a labeling module 920, an assignment engine module 925, and a communication module 940. In particular, the operating system 910 may manage the data processing system's software and/or hardware resources and may coordinate execution of programs by the processor. The featuring module 915 may be configured to perform one or more of the operations described above with respect to the featuring modules 225, 265, the flowcharts of FIGS. 3-5, and 7, and the charts of FIGS. 6A and 6B. The labeling module 920 may be configured to perform one or more of the operations described above with respect to the labeling module 230, the flowcharts of FIGS. 3-5, and 7, and the charts of FIGS. 6A and 6B. The assignment engine module 925 may comprise a machine learning engine module 930 and an AI engine module 935. The machine learning engine module 930 may be configured to perform one or more operations described above with respect to the machine learning engine 240, the flowcharts of FIGS. 3-5, and 7, and the charts of FIGS. 6A and 6B. The AI engine module 935 may be configured to perform one or more operations described above with respect to the AI engine 245, the flowcharts of FIGS. 3-5, and 7, and the charts of FIGS. 6A and 6B. The communication module 940 may be configured to support communication between, for example, the assignment engine server 140 and the assignment engine interface server 130 and/or the payors 110a, 110b, and 110c.

Although FIGS. 8-9 illustrate hardware/software architectures that may be used in data processing systems, such as the assignment engine server 140 of FIG. 1 and the data processing system 800 of FIG. 8, respectively, in accordance with some embodiments of the inventive concept, it will be understood that embodiments of the present invention are not limited to such a configuration but is intended to encompass any configuration capable of carrying out operations described herein.

Computer program code for carrying out operations of data processing systems discussed above with respect to FIGS. 1-9 may be written in a high-level programming language, such as Python, Java, C, and/or C++, for development convenience. In addition, computer program code for carrying out operations of the present invention may also be written in other programming languages, such as, but not limited to, interpreted languages. Some modules or routines may be written in assembly language or even micro-code to enhance performance and/or memory usage. It will be further appreciated that the functionality of any or all of the program modules may also be implemented using discrete hardware components, one or more application specific integrated circuits (ASICs), or a programmed digital signal processor or microcontroller.

Moreover, the functionality of the assignment engine server 140 of FIG. 1 and the data processing system 800 of FIG. 8 may each be implemented as a single processor system, a multi-processor system, a multi-core processor system, or even a network of stand-alone computer systems, in accordance with various embodiments of the inventive concept. Each of these processor/computer systems may be referred to as a “processor” or “data processing system.”

The data processing apparatus described herein with respect to FIGS. 1-9 may be used to facilitate AI assisted medical claim denial processing according to some embodiments of the inventive concept described herein. These apparatus may be embodied as one or more enterprise, application, personal, pervasive and/or embedded computer systems and/or apparatus that are operable to receive, transmit, process and store data using any suitable combination of software, firmware and/or hardware and that may be standalone or interconnected by any public and/or private, real and/or virtual, wired and/or wireless network including all or a portion of the global communication network known as the Internet, and may include various types of tangible, non-transitory computer readable media. In particular, the memory 905 when coupled to a processor includes computer readable program code that, when executed by the processor, causes the processor to perform operations including one or more of the operations described herein with respect to FIGS. 1-7.

Some embodiments of the inventive concept described herein may provide an AI assisted medical claim denial processing system that may prioritize medical claims that have been denied by payors in a manner that increases the likely return for the provider and/or patient. Moreover, the denied claims may be assigned to agents in an intelligent system using an AI system that takes into account characteristics of the agents including the agents' skillsets, availability, and/or location. This may further increase the likelihood that payor claim denials may be overcome and may increase the utilization efficiency of the staff that is available to pursue appeals of denied claims.

### Further Definitions and Embodiments

In the above description of various embodiments of the present inventive concept, it is to be understood that the terminology used herein is for the purpose of describing particular embodiments only and is not intended to be limiting of the invention. Unless otherwise defined, all terms (including technical and scientific terms) used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of this specification and the relevant art and will not be interpreted in an idealized or overly formal sense expressly so defined herein.

The flowchart and block diagrams in the figures illustrate the architecture, functionality, and operation of possible implementations of systems, methods, and computer program products according to various aspects of the present inventive concept. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular aspects only and is not intended to be limiting of the inventive concept. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Like reference numbers signify like elements throughout the description of the figures.

In the above-description of various embodiments of the present inventive concept, aspects of the present inventive concept may be illustrated and described herein in any of a number of patentable classes or contexts including any new and useful process, machine, manufacture, or composition of matter, or any new and useful improvement thereof. Accordingly, aspects of the present inventive concept may be implemented entirely hardware, entirely software (including firmware, resident software, micro-code, etc.) or combining software and hardware implementation that may all generally be referred to herein as a “circuit,” “module,” “component,” or “system.” Furthermore, aspects of the present inventive concept may take the form of a computer program product comprising one or more computer readable media having computer readable program code embodied thereon.

Any combination of one or more computer readable media may be used. The computer readable media may be a computer readable signal medium or a computer readable storage medium. A computer readable storage medium may be, for example, but not limited to, an electronic, magnetic, optical, electromagnetic, or semiconductor system, apparatus, or device, or any suitable combination of the foregoing. More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), an appropriate optical fiber with a repeater, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

The description of the present inventive concept has been presented for purposes of illustration and description, but is not intended to be exhaustive or limited to the inventive concept in the form disclosed. Many modifications and variations will be apparent to those of ordinary skill in the art without departing from the scope and spirit of the inventive concept. The aspects of the inventive concept herein were chosen and described to best explain the principles of the inventive concept and the practical application, and to enable others of ordinary skill in the art to understand the inventive concept with various modifications as are suited to the particular use contemplated.

What is claimed is:

1. A method, comprising:
receiving a plurality of payment denials for medical claims from a plurality of payors;
assigning, using an artificial intelligence engine, priorities to the plurality of payment denials, respectively; and
assigning, using the artificial intelligence engine, the plurality of payment denials to a plurality of agents responsible for obtaining payment approvals for the medical claims for which the payment denials have been received, respectively, based on the priorities.

2. The method of claim 1, wherein assigning, using the artificial intelligence engine, the priorities comprises:
assigning, using the artificial intelligence engine, the priorities to the plurality of payment denials, respectively, based on projected values associated with the plurality of payment denials, respectively.

3. The method of claim 2, further comprising:
determining, using the artificial intelligence engine, probabilities of obtaining payment approvals for the medical claims for which the payment denials have been received, respectively; and
estimating, using the artificial intelligence engine, payment amounts for the medical claims for which the payment denials have been received, respectively.

4. The method of claim 3, further comprising:
determining the projected values associated with the plurality of payment denials based on the probabilities of obtaining payment approvals that have been determined and the payment amounts that have been estimated, respectively.

5. The method of claim 1, wherein assigning, using the artificial intelligence engine, the plurality of payment denials to the plurality of agents comprises:
assigning, using the artificial intelligence engine, the plurality of payment denials to the plurality of agents based on the priorities and characteristics associated with each of the plurality of agents.

6. The method of claim 5, wherein the characteristics associated with each of the plurality of agents comprises one or more of a plurality of skillsets, an availability, and a location.

7. The method of claim 6, wherein each of the plurality of skillsets is associated with at least one of a plurality of claim adjustment reason codes used by the plurality of payors.

8. The method of claim 6, further comprising:
determining for one of the plurality of agents times taken for obtaining payment approvals for the medical claims that have been assigned to the one of the plurality of agents, respectively for different ones of the plurality of skillsets.

9. The method of claim 6, wherein the plurality of skillsets comprises a first skillset associated with provider credentialing, a second skillset associated with treatment pre-authorization, a third skillset associated with medical coding, a fourth skillset associated with payor plan eligibility, a fifth skillset associated with payor underpayment, a sixth skillset associated with documentation requests, a seventh high level general skillset, an eight medium level general skillset, and/or a ninth low level general skillset.

10. The method of claim 6, wherein the availability is based on a number of the plurality of payment denials assigned to the respective one of the plurality of agents.

11. The method of claim 6, wherein the location identifies a time zone for the respective one of the plurality of agents.

12. A system, comprising:
a processor; and
a memory coupled to the processor and comprising computer readable program code embodied in the memory that is executable by the processor to perform operations comprising:
receiving a plurality of payment denials for medical claims from a plurality of payors; and
assigning, using an artificial intelligence engine, priorities to the plurality of payment denials, respectively;
assigning, using the artificial intelligence engine, the plurality of payment denials to a plurality of agents responsible for obtaining payment approvals for the medical claims for which the payment denials have been received, respectively, based on the priorities.

13. The system of claim 12, wherein assigning, using the artificial intelligence engine, the priorities comprises:
assigning, using the artificial intelligence engine, the priorities to the plurality of payment denials, respectively, based on projected values associated with the plurality of payment denials, respectively.

14. The system of claim 13, wherein the operations further comprise:
determining, using the artificial intelligence engine, probabilities of obtaining payment approvals for the medical claims for which the payment denials have been received, respectively; and
estimating, using the artificial intelligence engine, payment amounts for the medical claims for which the payment denials have been received, respectively.

15. The system of claim 14, wherein the operations further comprise:
determining the projected values associated with the plurality of payment denials based on the probabilities of obtaining payment approvals that have been determined and the payment amounts that have been estimated, respectively.

16. The system of claim 12, wherein assigning, using the artificial intelligence engine, the plurality of payment denials to the plurality of agents comprises:
assigning, using the artificial intelligence engine, the plurality of payment denials to the plurality of agents based on the priorities and characteristics associated with each of the plurality of agents.

17. A computer program product, comprising:
a non-transitory computer readable storage medium comprising computer readable program code embodied in the medium that is executable by a processor to perform operations comprising:
receiving a plurality of payment denials for medical claims from a plurality of payors;
assigning, using an artificial intelligence engine, priorities to the plurality of payment denials, respectively; and
assigning, using the artificial intelligence engine, the plurality of payment denials to a plurality of agents responsible for obtaining payment approvals for the medical claims for which the payment denials have been received, respectively, based on the priorities.

18. The computer program product of claim 17, wherein assigning, using the artificial intelligence engine, the priorities comprises:
assigning, using the artificial intelligence engine, the priorities to the plurality of payment denials, respectively, based on projected values associated with the plurality of payment denials, respectively;
wherein the operations further comprise:
determining, using the artificial intelligence engine, probabilities of obtaining payment approvals for the medical claims for which the payment denials have been received, respectively; and
estimating, using the artificial intelligence engine, payment amounts for the medical claims for which the payment denials have been received, respectively.

19. The computer program product of claim 18, wherein the operations further comprise:
determining the projected values associated with the plurality of payment denials based on the probabilities of obtaining payment approvals that have been determined and the payment amounts that have been estimated, respectively.

20. The computer program product of claim 17, wherein assigning, using the artificial intelligence engine, the plurality of payment denials to the plurality of agents comprises:
assigning, using the artificial intelligence engine, the plurality of payment denials to the plurality of agents based on the priorities and characteristics associated with each of the plurality of agents.
